(19) United States
(12) Patent Application Publication
(10) Pub. No.: US 2006/0058854 A1
(43) Pub. Date: Mar. 16, 2006

(54) Method for stimulating neural tissue in response to a sensed physiological event

(75) Inventors: Robert Abrams, Los Gatos, CA (US); Michael P. Wallace, Pleasanton, CA (US); Robert J. Garabedian, Mountain View, CA (US)

Correspondence Address: Bingham McCuthen, LLP, Suite 1800, Three Embarcadero, San Francisco, CA, 94111-4067, US

(73) Assignee: Scimed Life Systems, Inc., Maple Grove, MN (US)

(21) Appl. No.: 10/941,382

(22) Filed: Sep. 14, 2004

(51) Int. Cl. A61N 1/18

(52) U.S. Cl. 607/45

(57) ABSTRACT

A method of treating a disorder of a patient, such as a neurological disorder, is provided. The method comprises intravascularly delivering at least one of a stimulation lead and a sensing lead within the head of the patient. For example, one or both of the leads can be delivered into the patient's head via the circulatory system (e.g., vein or artery) or the ventricular system (e.g., through the intrathecal space of the spine). The method further comprises sensing a physiological event associated with the disorder (e.g., physiological electrical activity, a blood parameter, or intracranial pressure) using the sensing lead, and stimulating neural tissue with the stimulation lead in response to the sensed physiological event.

### FIELD OF THE INVENTION

The invention relates to the treatment and diagnosis of physiological disorders, and in particular, the treatment and diagnosis of physiological disorders using electrical stimulation.

### BACKGROUND OF THE INVENTION

It is sometimes desirable to treat disorders in patients using implantable feedback electrical stimulation systems. For example, neurological disorders, such epilepsy, migraine headaches, and Parkinson's disease, can be treated by sensing a neurological event and then electrically stimulating a selected region of the patient's brain (such as the cortex or deep brain) using stimulation leads to control the disorder. See, e.g., U.S. Pat. No. 6,016,449, which is expressly incorporated herein by reference. In these cases, access to the patient's brain is accomplished using a fairly invasive procedure, which involves drilling burr holes through the patient's cranium or performing a craniotomy on the patient in order to gain access to the brain tissue. In the case of deep brain stimulation, the stimulation lead must be introduced through the parenchyma of the brain until it reaches the desired stimulation site within the selected deep brain structure. Sensing leads, used to sense EEG signals from the brain can then be implanted within the brain via one or more burr holes or a craniotomy or within the scalp of the patient.

Once the stimulation lead is properly located in contact with the selected brain tissue, the proximal end of the lead or an extension lead is subcutaneously routed from the burr hole underneath the patient's scalp, down the neck, and into the chest or abdominal region of the patient in electrical connection with an implanted electrical stimulator. The sensing lead will also be subcutaneously routed, either from a burr hole if located in or on the patient's brain, or from the scalp, and connected to the implanted electrical stimulator. The electrical stimulator is programmed either prior to or after the procedure to deliver electrical pulses to the brain tissue via the stimulation lead. The electrical stimulator has feedback control, in which case, the onset of an epileptic seizure can be predicted by sensing the EEG signals with the sensing lead, and transmitting the sensed EEG signals back to the electrical stimulator, which responds by transmitting stimulation signals to the stimulation lead as necessary.

Although the current brain stimulation/feedback techniques used to treat neurological disorders have proven to be successful, such techniques are still quite invasive, requiring the cranium to be opened through a burr hole or craniotomy. In addition, the need for a burr hole further complicates the procedure-not only requiring the additional step of accessing the patient's cranium while attempting to minimize tissue trauma, but also requiring that the burr hole be capped at the end of the procedure. Also, additional risks are posed by the possibility that the burr hole may become infected and the routing of the stimulation or extension leads through the neck in close proximity to the jugular veins and carotid arteries.

Thus, there remains a need to provide improved methods for delivering stimulation and/or sensing leads within a patient's head to treat physiological disorders in response to sensed physiological events.

### SUMMARY OF THE INVENTION

In accordance with the present invention, a method of treating a disorder of a patient is provided. The disorder may be of any nature. For example, the disorder may be neurological, in which case, the disorder may be a neurodegenerative disorder, such as Parkinson's disease, Huntington's disease, or Alzheimer's disease, or a non-neurodegenerative disorder, such as epilepsy, essential tremor, cerebral ischemia, vasospasm, depression, obsessive compulsive disorder, schizophrenia, and neuropathic pain. The disorder may also be non-neurological, for example, drug refractory hypertension.

The method comprises delivering a stimulation lead into the head of the patient, and placing it adjacent tissue (e.g., brain tissue or nerve tissue)—either in direct contact or indirect contact with the tissue. Depending on the disorder to be treated, the stimulation lead may be placed, e.g., either acutely, subchronically, or chronically, within the patient's head.

The method further comprises sensing a physiological event associated with the disorder using a sensing device, and stimulating the tissue with the stimulation lead in response to the physiological event. For example, the onset or occurrence of a disorder may be indicated by a change in electrical activity (e.g., a change in electrical activity in the patient's cerebral or nerve tissue), in which case, the physiological event to be sensed is a change in physiological electrical activity. Or the onset or occurrence of a disorder may be indicated by a change in a blood parameter (such as blood pressure, blood flow, heart rate, oxygen level, etc.) or a change in intracranial pressure, in which case, the physiological event to be sensed is a change in the blood parameter or intracranial pressure.

The physiological parameter may be, e.g., sensed at a location adjacent the patient's head or at a location peripheral from the patient's head, and may be sensed at a location that is either internal or external to the patient's body. The method may optionally comprise intravascularly delivering a sensing lead within the patient, in which case, the sensing lead can be used to sense the physiological event. To minimize the invasiveness of the procedure, one or both of the stimulation lead and sensing device are intravascularly delivered within the patient, for example, via the circulatory or ventricular system.

In the case where the stimulation lead is intravascularly delivered within the patient's head, the method may comprise making an access point into the vasculature of the patient (e.g., within the circulatory or ventricular system of the patient), introducing the stimulation lead through the access point and into the head of the patient, and placing the stimulation lead adjacent brain tissue within the head, while the proximal end of the stimulation lead extends from the access point. The proximal end of the stimulation lead and the sensing device can be connected to a stimulation source configured to transmit stimulation energy to the stimulation lead in response to a feedback signal from the sensing device. The stimulation source may be implanted within the patient external to the vasculature of the patient. The method may optionally comprise conveying stimulation energy from the stimulation source to the stimulation lead in order to electrically stimulate the brain tissue to treat the disorder.

### BRIEF DESCRIPTION OF THE DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view of an intravascular brain stimulation system constructed in accordance with a preferred embodiment of the present invention.

### DETAILED DESCRIPTION OF THE PREFERRED EMBODIMENTS

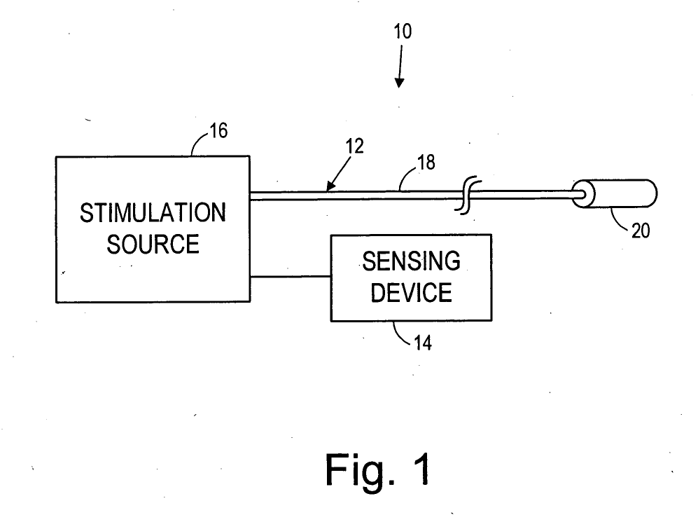

Referring now to FIG. 1, an intravascular brain stimulation system 10 constructed in accordance with one preferred embodiment of the present invention is shown. Depending on its specific arrangement within a patient's body, the stimulation system 10 is configured for treating a specific disorder from which the patient suffers by stimulating a specific region of the patient's brain in response to a sensed physiological event associated with the specific disorder. For example, the system 10 can be designed treat neurodegenerative disorders, such as Parkinson's disease, Huntington's disease, Alzheimer's disease, or other chronic neurological disorders, such as epilepsy, essential tremor, depression, obsessive compulsive disorder, schizophrenia, and neuropathic pain. In these cases, the sensed physiological event used to trigger stimulation of the patient's brain can be, e.g., a change in electrical activity in the patient's cerebral or nerve tissue, which may indicate the onset or occurrence of the symptoms of the disorder. The system 10 can also be designed to treat acute neurological disorders, such as cerebral ischemia, which is typically caused by a stroke, or cerebral vasospasm, which is typically caused by bleeding of the brain. The system 10 may even be designed to treat non-neurological disorders, such as drug refractory hypertension. In these cases, the sensed physiological event used to trigger stimulation of the patient's brain can be, e.g., a change in a blood parameter, such as cerebral or peripheral blood pressure, cerebral blood flow, heart rate, or cerebral blood oxygen level, or a change in some other physiological parameter, such as intracranial pressure.

In its simplest form, the stimulation system 10 generally comprises a stimulation lead 12 configured for electrically stimulating a selected region of a patient's brain, a sensing device 14 configured for sensing the physiological event, and an implantable electrical stimulation source 16 configured for receiving electrical feedback signals from the sensing device 14, and in response thereto, transmitting electrical stimulation energy to the stimulation lead 12 to treat the disorder with which the sensed physiological event is associated. In alternative embodiments, multiple stimulation leads 12 and/or multiple sensing devices 14 can be provided, depending on the size and locations of the selected brain regions to be stimulated.

The stimulation lead 12 is configured to be intravascularly placed within the patient. To this end, the stimulation lead 12 comprises a flexible electrically conductive signal wire 18 and a single electrode 20 mounted at the distal end of the wire 18 using suitable connection means, such as soldering or welding. In the illustrated embodiment, the electrode 20 is cylindrically shaped and has a size that allows it to be delivered through a delivery catheter. The wire 18 comprises an electrically conductive core with an outer insulative layer. The length of the wire 18 is preferably sized to extend from the selected stimulation site in the brain to the implant location of the stimulation source 16. For example, if the stimulation source 16 is to be implanted in the chest region of the patient, the length of the wire 18 may be in the range of 50 cm to 100 cm. If, however, the stimulation source 16 is to be implanted in the abdomen or groin area of the patient, the length of the wire 18 may be in the range of 150 cm to 300 cm. The electrode 20 is composed of a biocompatible and electrically conducting material, such as copper alloy, platinum, stainless steel, or nitinol. The electrically conducting material of the electrode 20 can be further coated with platinum-iridium or gold to improve its conduction properties, biocompatibility, and radiopacity. To prevent blood clotting, the electrode lead 12 can be optionally coated with a non-thrombogenic agent. The electrode 12 can also be coated with a therapeutic drug to prevent tissue in-growth or to minimize scar tissue around the electrode 12, thereby allowing the electrode 12 to be more easily removed from the patient if desired.

It should be noted that the intravascular stimulation lead 12 may have a different structure than that illustrated in FIG. 1. For example, the intravascular stimulation lead 12 may alternatively or optionally have multiple electrodes (e.g., for bipolar or multipolar operation), stent electrode, arrayed electrode structure, basket electrode structure, inflatable electrode structure, helical electrode structure, etc., may take the form of a guidewire or catheter, and may have optional blood occlusion features, such as a balloon or RF ablation electrode, the details of which are disclosed in U.S. patent application Ser. No. 10/744,319, entitled “Method of Intravascularly Delivering Stimulation Leads into the Brain”, which is expressly incorporated herein by reference.

Optionally, a non-vascular stimulation lead (not shown) can be used in combination with the intravascular stimulation lead 12, e.g., to stimulate brain tissue regions too remote to stimulate from vascular bodies. A system for stimulating brain tissue using a combination of intravascular and non-intravascular stimulation leads is described in U.S. patent application Ser. No. 10/783,679, entitled “Method of Stimulating/Sensing Brain with Combination of Intravascularly and Non-Vascularly Delivered Leads,” which is expressly incorporated herein by reference.

The design and functionality of the sensing device 14 will ultimately depend on the nature and location of the physiological event to be sensed. For example, if the physiological event to be sensed is electrical brain activity (e.g., to sense electroencephalogram (EEG) signals), the sensing device 14 may take the form of a sensing lead that is configured to be intravascularly placed within the patient's head—much like the stimulation lead 12 described above. In this case, the sensing lead may comprise one or more sensing elements in the form of electrodes for sensing the electrical activity within the patient's brain. Alternatively, the sensing device 14 may take the form of an electrode paddle or array that can be located over the dura of the brain to sense electrical signals within the cortical brain tissue—although this may be somewhat more invasive than the use of an intravascular lead. The sensing device 14 may even take the form of external electrodes placed on or in the scalp of the patient. If the physiological event to be sensed in electrical nerve activity (e.g., to sense electromyogram (EMG) signals), the sensing device 14 may take the form of a nerve cuff that can be placed around the spinal cord or a peripheral motor nerve, a subdermal implant, or even needle or surface electrodes to sense afferent nerve activity, which may indicate, e.g., the occurrence of involuntary movements caused by such disorders as Parkinson's disease, essential tremor, and epilepsy.

If the physiological event to be sensed in a blood parameter, the sensing device 14 may take the form of a sensing lead that is configured to be intravascularly placed within the patient's head—much like the stimulation lead 12 described above. In this case, the sensing lead may take one or more sensing elements in the form of a Doppler ultrasound transducer for sensing blood flow, or a pressure sensor for sensing blood pressure. Alternatively, the sensing device 14 may take the form of an extravascular probe that can be used to sense the blood parameter within a blood vessel using suitable means, such as ultrasound. Or, the sensing device 14 can be an external device, such as a heart monitor. The blood parameter can be sensed at a location within the patient's brain (e.g., the blood pressure, blood flow, or blood oxygen level may drop in a particular region of the patient's brain as a result of cerebral ischemia or vasospasm) or may be sensed at a peripheral location (e.g., the blood pressure may systemically increase in response to drug refractory hypertension, in which case, the blood pressure may be measured anywhere in the patent's body). If the physiological event to be sensed is intracranial pressure, which may indicate the presence of cerebral ischemia or vasospasm, the sensing device may be a brain tissue probe (although somewhat more invasive) or an intraventricular brain probe.

Referring still to FIG. 1, the implantable stimulation source 16 is designed to deliver electrical pulses to the stimulation lead 12 in accordance with programmed parameters. In the preferred embodiment, the stimulation source 16 is programmed to output electrical pulses having amplitudes varying from 0.1 to 20 volts, pulse widths varying from 0.02 to 1.5 milliseconds, and repetition rates varying from 2 to 2500 Hertz. In the illustrated embodiment, the stimulation source 16 takes the form of a totally self-contained generator, which once implanted, may be activated by a small magnet and/or controlled by an outside telemetry source that transmits programmed parameters to the pulse generator and monitors the performance of the pulse generator, e.g., a small magnet. In this case, the pulse generator has an internal power source that limits the life of the pulse generator to a few years, and after the power source is expended, the pulse generator must be replaced. Generally, these types of stimulation sources 16 may be implanted within the chest or abdominal region beneath the skin of the patient.

Alternatively, the implantable stimulation source 16 may take the form of a passive receiver that receives radio frequency (RF) signals from an external transmitter worn by the patient. In this scenario, the life of the stimulation source 16 is virtually unlimited, since the stimulation signals originate from the external transmitter. Like the self-contained generators, the receivers of these types of stimulation sources 16 can be implanted within the chest or abdominal region beneath the skin of the patient. The receivers may also be suitable for implantation behind the ear of the patient, in which case, the external transmitter may be worn on the ear of the patient in a manner similar to that of a hearing aid. Stimulation sources, such as those just described, are commercially available from Medtronic, Inc., located in Minneapolis, Minn. Further details regarding the construction of a stimulation source for the purpose of treating neurological disorders is disclosed in U.S. Pat. No. 5,716,377, which is expressly incorporated herein by reference.

The stimulation source 16 may be connected to the stimulation lead 12 or multiple stimulation leads 12 in any one of a variety of manners. For example, each stimulation lead 12 can be connected in a unipolar arrangement or a bipolar or multipolar arrangement (if the lead 12 carries multiple electrodes), or multiple stimulation leads 12 can be connected together in a bipolar arrangement, further details of which are described in U.S. patent application Ser. No. 10/744,319, which has previously be incorporated herein by reference. The stimulation source 16 may be connected to the sensing device 14 in any suitable manner.

Having described the construction and function of the brain stimulation system 10, a preferred method of installing it within a patient's body in order to treat a diagnosed disorder will now be described. The stimulation lead 12 will be intravascularly introduced within the patient's head adjacent a selected brain region. The routing and placement of the brain stimulation system 10 will ultimately depend on the portion of the brain that is to be treated. For example, the cortex of the brain or the deep brain can be electrically stimulated to treat Parkinson's disease, essential tremor, Huntington's disease, Alzheimer's disease, epilepsy, depression, obsessive compulsive disorder, schizophrenia, and neuropathic pain. Any lobe of the cortex or deep brain can be stimulated. Preferably, for the cortical region of the brain, the motor strip, sensor strip, and premotor cortex should be stimulated.

For the deep brain region, the anterior thalamus, ventrolateral thalamus (Thai), internal segment of globus pallidus (GPi), substantia nigra pars reticulata (SNr), subthalamic nucleus (STN), external segment of globus pallidus (GPe), neostriatum, cingulate, and cingulate gyrus should be stimulated. Certain hindbrain structures, such as the fastigium nucleus (FN), which can control the amount of blood flow to the brain, may also be stimulated, e.g., to hyperperfuse a hemisphere of the brain damaged as a result of an ischemic event, such as a stroke, or to help metabolize amlyoid plaques caused by Alzheimer's Disease and prevent the occurrence of vasospasms, both achieved through increased blood flow to the brain. A method of stimulating the FN and other hindbrain structures to increase blood flow within the select regions of the brain is described in U.S. patent application Ser. No. 10/893,076, entitled “Method of Stimulating Fastigium Nucleus to Treat Neurological Disorders,” which is expressly incorporated herein by reference. Certain ganglia, such as the spheno palatine ganglion (SPG), may also be stimulated to hyperperfuse select regions of the brain.

In order to minimize the invasiveness of the procedure, at least a portion of the preexisting vasculature (e.g., the circulatory or ventricular system) of the patient is utilized to gain access to the stimulation site. This can be accomplished, e.g., by first introducing the stimulation lead into the patient's head via the selected vascular system and then advancing the stimulation lead within selected cerebral vascular bodies within that system until the active portion of the stimulation lead is adjacent the selected stimulation site, or alternatively, by first non-vascularly introducing the stimulation lead into the patient's head (e.g., through a burr hole in the cranium) and then advancing the stimulation lead within vascular bodies of a selected vascular system until the stimulation lead is adjacent the selected stimulation site. The former case is less invasive than the latter case, since it entirely uses the vasculature to both introduce the stimulation lead into the patient's head and, once inside the head, deliver the stimulation lead to a site adjacent the selected stimulation site. Additional stimulation leads can optionally be placed adjacent the same or other stimulation sites as necessary.

A standard imaging system, such as Computed Tomography Angiography (CTA), fluoroscopy, Magnetic Resonance Imaging (MRI), and/or ultrasound, and a standard delivery mechanism, such as a guide wire, delivery catheter, and/or guide sheath (all not shown), can be used to facilitate delivery of the stimulation lead into the patient's head and/or route the stimulation lead to a location adjacent the selected stimulation site. Of course, if the stimulation lead, itself takes the form of a guidewire or catheter, a separate guide wire or catheter may not be needed. The stimulation lead may be maintained within the vascular body adjacent the selected stimulation site, such that stimulation can be indirectly applied to the selected stimulation site, or alternatively, can be inserted through a puncture within the vascular body into direct contact with the selected stimulation site with the aid of a stylet. Details describing various methods for placing intravascularly delivered stimulation leads into direct contact with brain tissue are disclosed in U.S. patent application Ser. No. 10/744,853, entitled “Method of Intravascularly Delivering Stimulation Leads into Direct Contact with Tissue,” which is expressly incorporated herein by reference.

If the stimulation lead has an anchoring capability (e.g., it has as a stent electrode, arrayed electrode structure, basket electrode structure, inflatable electrode structure, helical electrode structure, etc.), the stimulation lead can be deployed in order to stabilize the stimulation lead relative to the FN. Further details describing the delivery and deployment of stimulation leads into indirect or direct contact with brain tissue are provided in U.S. patent application Ser. No. 10/744,319, which has previously been incorporated herein by reference.

The stimulation lead 12 can be delivered to any one of a number of vessels in order to place the active portion of the stimulation lead adjacent the cortical tissue to be stimulated. Examples of veins providing access to the cortex include the superior sagittal sinus, any of the superior cerebral veins branching from the superior sagittal sinus (e.g., the lacuna, frontopolar vein, anterior frontal vein, posterior frontal vein, precentral vein, central vein, anterior parietal vein, posterior parietal vein, and occipital vein), superior sylvian vein, vein of Labbe, vein of Trolard, inferior sagittal sinus, and any inferior cerebral veins branching off of the inferior sagittal sinus, transverse sinus, and meningeal sinus. Examples of arteries providing access to the cortex include any of the branches off of the external carotid, maxillary, or meningeal arteries.

Examples of veins providing access to the deep brain include the inferior sagittal sinus, pericallosal sinus, cavernous sinus, sphenoid sinus, temperal basal vein, and occipital veins. Examples of arteries providing access to the deep brain include any branches off of the internal carotid or vertebral arteries. Examples of veins providing access to the SPG include the superficial temporal veins and the facial vein. Examples of arteries providing access to the SPG include the maxillary artery, descending palatine artery, and facial artery.

Examples of arteries providing access to a hindbrain structure, such as the FN, includes the posterior inferior cerebellar artery (PICA), or an artery branching from the PICA. Examples of veins providing access to the FN include the vein of galen, superior cerebellar vein, and preculiminate vein. The hindbrain structure can also be access through the ventricular system, e.g., through the 4^{th} ventricle.

The jugular and femoral veins can be used as intrasvascular access points from which stimulation lead can be delivered to the above-described veins, and the carotid, femoral, and vertebral arteries can be used as intrasvascular access points from which the stimulation lead can be delivered to the above-described arteries. The intrathecal space can be used as an intravascular access point from which the stimulation lead can be delivered to the 4^{th} ventricle.

Of course, in those brain regions that are not adjacent one of these blood vessels or ventricular bodies, or is otherwise adjacent a blood vessel or ventricular body that is not navigatable by the stimulation lead 12, such brain regions will have to be reached by non-vascular means, e.g., by epidurally or subdurally placing the stimulation lead along the cortex for cortical simulation, or by penetrating the parenchyma for deep brain stimulation.

In some situations, it may not be necessary to deliver the stimulation lead 12 within the patient's head. For example, some disorders, such as chronic pain, can be treated by electrically stimulating nerve tissue, such as the spinal cord, vagus nerve, or trigeminal nerve. In this case, the stimulation lead 12 can be implanted within the epidural or intrathecal space of the spine, or can be implanted within a portion of the patient's body, e.g., the neck region, where access to the vagus or trigeminal nerve is provided.

After the stimulation lead 12 has been deployed within the patient, its proximal end will remain outside of the patient's body after the stimulation deployment process is completed. For example, if the stimulation lead 12 is intravascularly introduced into the patient via the circulatory system, the proximal end stimulation lead 12 will extend from a venous or arterial access point. If the stimulation lead 12 is intravascularly introduced into the patient via the ventricular system, the proximal end of the stimulation lead 12 will extend from the intrathecal space of the patient's spine. If the stimulation lead 12 is introduced into the patient's head via a burr hole, the proximal end of the cranial burr hole will extend from the burr hole.

The exposed proximal end of the stimulation lead 12 can be subcutaneously routed a short distance to the clavical or chest region or behind the ear of the patient (in this case where the jugular vein is the access point) or the abdominal or groin region of the patient (in the case where the femoral vein is the access point), where they can be coupled to the stimulation source 16. The stimulation source 16 will then be implanted within the patient's body (e.g., in the clavical or chest region or behind the ear of the patient).

The sensing device 14 can be associated within the patient's body in any one of a variety of manners. For example, if the sensing device 14 takes the form of an intravascular lead, it can be intravascularly delivered into the patient's head in the same manner described above with respect to the stimulation lead 12, and placed into direct or indirect contact with the region of the body to be sensed, e.g., the blood within a blood vessel or a selected region of the brain. If the sensing device 14 takes the form of a peripheral device, it can be implanted within or otherwise located at the peripheral region to be sensed. If the sensing device 14 takes the form of an external sensing device it can be appropriately attached to the skin of the patient's body. In any event, once properly located, the sensing device 14 can be coupled to the stimulation source 16 to provide sensory feedback. The stimulation source 16 may then be operated to provide electrical stimulation energy to the selected stimulation site of the patent's body in response to the feedback provided by the sensing device 14. In an optional embodiment, the control parameters of the stimulation source 16 may be selected or changed based on the feedback signals transmitted by the sensing device 14. For example, if the disorder is a vasospasm, the intensity of the stimulation energy transmitted by the stimulation source 16 to the stimulation lead 12 may need to be adjusted based upon blood pressure sensed by the sensing device 14 in order to improve the safety or efficacy of the system 10.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method of treating a disorder of a patient, comprising:
intravascularly delivering a stimulation lead within the head of the patient;
placing the stimulation lead adjacent neural tissue;
sensing a physiological event associated with the disorder; and
stimulating the neural tissue with the stimulation lead in response to the sensed physiological event.

2. The method of claim 1, wherein the disorder is a neurological disorder.

3. The method of claim 1, wherein the stimulation lead is introduced into the head via the circulatory system.

4. The method of claim 1, wherein the stimulation lead is introduced into the head via the ventricular system.

5. The method of claim 1, wherein the stimulation lead is placed in direct contact with the neural tissue.

6. The method of claim 1, wherein the stimulation lead is placed in indirect contact with the neural tissue.

7. The method of claim 1, wherein the neural tissue is brain tissue.

8. The method of claim 1, wherein the neural tissue is nerve tissue.

9. The method of claim 1, wherein the stimulation lead is acutely placed adjacent the neural tissue.

10. The method of claim 1, wherein the stimulation lead is subchronically or chronically placed adjacent the neural tissue.

11. The method of claim 1, further comprising intravascularly delivering a sensing lead within the patient, wherein the sensing lead senses the physiological event.

12. The method of claim 1, further comprising intravascularly delivering a sensing lead within the head of the patient, wherein the sensing lead senses the physiological event.

13. The method of claim 1, wherein the physiological event sensing comprises sensing a parameter at a location adjacent the head of the patient.

14. The method of claim 1, wherein the physiological event sensing comprises sensing a parameter at a location peripheral from the head of the patient.

15. The method of claim 1, wherein the physiological event sensing comprises sensing a parameter from a location internal to the patient.

16. The method of claim 1, wherein the physiological event sensing comprises sensing a parameter from a location external to the patient.

17. The method of claim 1, wherein the physiological event sensing comprises sensing physiological electrical activity.

18. The method of claim 1, wherein the physiological event sensing comprises sensing intracranial pressure.

19. The method of claim 1, wherein the physiological event sensing comprises sensing a blood parameter.

20. The method of claim 1, wherein the disorder comprises at least one of Parkinson's disease, Alzheimer's disease, Huntington's disease, epilepsy, essential tremor, depression, obsessive compulsive disorder, schizophrenia, and neuropathic pain, and the sensed physiological event is a change in electrophysiological electrical activity.

21. The method of claim 1, wherein the disorder comprises at least one of cerebral ischemia, cerebral vasospasm, and refractory hypertension, and the sensed physiological event is at least one of a change in blood flow, a change in blood pressure, a change in the blood oxygen level, and a change in intracranial pressure.

22. A method of treating a neurological disorder of a patient, comprising:
delivering a stimulation lead within the head of the patient;
placing the stimulation lead adjacent neural tissue;
sensing a physiological event associated with the neurological disorder using a sensing device, wherein at least one of the stimulation lead and sensing device is intravascularly delivered within the patient; and
stimulating the neural tissue with the stimulation lead in response to the sensed physiological event.

23. The method of claim 22, wherein at least one of the stimulation lead and sensing device is introduced into the patient via the circulatory system.

24. The method of claim 22, wherein at least one of the stimulation lead and sensing device is introduced into the patient via the ventricular system.

25. The method of claim 22, wherein the stimulation lead is placed in direct contact with the neural tissue.

26. The method of claim 22, wherein the stimulation lead is placed in indirect contact with the neural tissue.

27. The method of claim 22, wherein the neural tissue is brain tissue.

28. The method of claim 22, wherein the neural tissue is nerve tissue.

29. The method of claim 22, wherein the stimulation lead is acutely placed adjacent the neural tissue.

30. The method of claim 22, wherein the stimulation lead is subchronically or chronically placed adjacent the neural tissue.

31. The method of claim 22, wherein the sensing device comprises a sensing lead that is intravascularly delivered into the patient.

32. The method of claim 22, wherein the sensing lead is intravascularly delivered into the patient.

33. The method of claim 22, wherein the physiological event sensing comprises sensing a parameter at a location adjacent the head of the patient.

34. The method of claim 22, wherein the physiological event sensing comprises sensing a parameter at a location peripheral from the head of the patient.

35. The method of claim 22, wherein the physiological event sensing comprises sensing a parameter from a location internal to the patient.

36. The method of claim 22, wherein the physiological event sensing comprises sensing a parameter from a location external to the patient.

37. The method of claim 22, wherein the physiological event sensing comprises sensing physiological electrical activity.

38. The method of claim 22, wherein the physiological event sensing comprises sensing intracranial pressure.

39. The method of claim 22, wherein the physiological event sensing comprises sensing a blood parameter.

40. The method of claim 22, wherein the neurological disorder comprises at least one of Parkinson's disease, Alzheimer's disease, Huntington's disease, epilepsy, essential tremor, depression, obsessive compulsive disorder, schizophrenia, and neuropathic pain, and the sensed physiological event is a change in electrophysiological electrical activity.

41. The method of claim 22, wherein the neurological disorder comprises at least one of cerebral ischemia, cerebral vasospasm, and refractory hypertension, and the sensed physiological event is at least one of a change in blood flow, a change in blood pressure, a change in the blood oxygen level, and a change in intracranial pressure.

42. A method of treating a disorder in a patient, comprising:
making an access point into the vasculature of the patient;
introducing a stimulation lead through the access point and into the head of the patient;
placing the stimulation lead adjacent neural tissue within the head, while a proximal end of the stimulation lead extends from the access point;
connecting the proximal end of the stimulation lead to a stimulation source, wherein the stimulation source is configured to transmit stimulation energy to the stimulation lead in response to a feedback signal; and
connecting a sensing device to the stimulation source, wherein the sensing device is configured to sense a physiological event and transmit the feedback signal to the stimulation source.

43. The method of claim 42, wherein the disorder is a neurological disorder.

44. The method of claim 42, wherein the access point is made in the circulatory system of the patient.

45. The method of claim 42, wherein the access point is made in the ventricular system of the patient.

46. The method of claim 42, wherein the stimulation lead is placed in direct contact with the neural tissue.

47. The method of claim 42, wherein the stimulation lead is placed in indirect contact with the neural tissue.

48. The method of claim 42, wherein the neural tissue is brain tissue.

49. The method of claim 42, wherein the neural tissue is nerve tissue.

50. The method of claim 42, wherein the stimulation lead is acutely placed adjacent the neural tissue.

51. The method of claim 42, wherein the stimulation lead is subchronically or chronically placed adjacent the neural tissue.

52. The method of claim 42, wherein the physiological event sensing comprises sensing a parameter at a location adjacent the head of the patient.

53. The method of claim 42, wherein the physiological event sensing comprises sensing a parameter at a location peripheral from the head of the patient.

54. The method of claim 42, wherein the physiological event sensing comprises sensing a parameter from a location internal to the patient.

55. The method of claim 42, wherein the physiological event sensing comprises sensing a parameter from a location external to the patient.

56. The method of claim 42, wherein the physiological event sensing comprises sensing physiological electrical activity.

57. The method of claim 42, wherein the physiological event sensing comprises sensing intracranial pressure.

58. The method of claim 42, wherein the physiological event sensing comprises sensing a blood parameter.

59. The method of claim 42, wherein the disorder comprises at least one of Parkinson's disease, Alzheimer's disease, Huntington's disease, epilepsy, essential tremor, depression, obsessive compulsive disorder, schizophrenia, and neuropathic pain, and the sensed physiological event is a change in electrophysiological electrical activity.

60. The method of claim 42, wherein the disorder comprises at least one of cerebral ischemia, cerebral vasospasm, and refractory hypertension, and the sensed physiological event is at least one of a change in blood flow, a change in blood pressure, a change in the blood oxygen level, and a change in intracranial pressure.

61. The method of claim 42, further comprising implanting the stimulation source within the patient external to the vasculature of the patient.

62. The method of claim 42, further comprising conveying stimulation energy from the stimulation source to the stimulation lead, wherein the neural tissue is electrically stimulated to treat the disorder.

63. The method of claim 62, further comprising sensing the physiological event with the sensing device.

64. The method of claim 42, further comprising modifying a stimulation parameter within the stimulation source based on the sensed physiological event.

65. A method of treating a disorder of a patient, comprising:
intravascularly delivering at least one of a stimulation lead and a sensing lead within the head of the patient;
sensing a physiological event associated with the disorder using the sensing lead; and
stimulating neural tissue with the stimulation lead in response to the sensed physiological event.

66. The method of claim 65, wherein the disorder is a neurological disorder.

67. The method of claim 65, wherein the at least one stimulation lead and sensing lead is introduced into the head via the circulatory system.

68. The method of claim 65, wherein the at least one stimulation lead and sensing lead is introduced into the head via the ventricular system.

69. The method of claim 65, wherein the physiological event sensing comprises sensing physiological electrical activity.

70. The method of claim 65, wherein the physiological event sensing comprises sensing intracranial pressure.

71. The method of claim 65, wherein the physiological event sensing comprises sensing a blood parameter.

72. The method of claim 65, wherein the disorder comprises at least one of Parkinson's disease, Alzheimer's disease, Huntington's disease, epilepsy, essential tremor, depression, obsessive compulsive disorder, schizophrenia, and neuropathic pain, and the sensed physiological event is a change in electrophysiological electrical activity.

73. The method of claim 65, wherein the disorder comprises at least one of cerebral ischemia, cerebral vasospasm, and refractory hypertension, and the sensed physiological event is at least one of a change in blood flow, a change in blood pressure, a change in the blood oxygen level, and a change in intracranial pressure.

74. The method of claim 65, wherein the at least one of a stimulation lead and a sensing lead comprises the stimulation lead.

75. The method of claim 65, wherein the at least one of a stimulation lead and a sensing lead comprises the sensing lead.

76. The method of claim 65, wherein the at least one of a stimulation lead and a sensing lead comprises both the stimulation lead and the sensing lead.
